(19) United States
(12) Patent Application Publication
(10) Pub. No.: US 2022/0155322 A1
(43) Pub. Date: May 19, 2022

(54) USE OF NATRIURETIC PEPTIDES TO ASSESS AND TREAT ACUTE KIDNEY INJURY

(71) Applicant: Mayo Foundation for Medical Education and Research, Rochester, MN (US)

(72) Inventors: Sasantha J. Sangaralingham, Rochester, MN (US); Yang Chen, Rochester, MN (US); John C. Burnett, Jr., Rochester, MN (US)

(21) Appl. No.: 17/433,012

(22) PCT Filed: Mar. 6, 2020
(86) PCT No.: PCT/US2020/021363

§ 371 Date: Aug. 23, 2021

Related U.S. Application Data
(60) Provisional application No. 62/819,088, filed on Mar. 15, 2019.

(51) Int. Cl. G01N 33/68

(52) U.S. Cl. CPC G01N 33/6893; G01N 2800/347; G01N 2333/58

(57) ABSTRACT

This document provides methods and materials related to using natriuretic peptides (NPs) as markers for acute kidney injury (AKI), and methods and materials for using NPs to treat patients identified as having, or being likely to have, AKI.

### CROSS-REFERENCE TO RELATED APPLICATIONS

This application claims benefit of priority from U.S. Provisional Application Ser. No. 62/819,088, filed Mar. 15, 2019.

### STATEMENT AS TO FEDERALLY SPONSORED RESEARCH

This invention was made with government support under HL036634 and HL132854 awarded by the National Institutes of Health. The government has certain rights in the invention.

### TECHNICAL FIELD

This document relates to natriuretic polypeptides (NPs). For example, this document provides methods and materials related to using NPs as markers for acute kidney injury (AKI), and using NPs to treat patients identified as having, or being likely to have, AKI.

### BACKGROUND

AKI is a powerful risk factor for incident heart failure (HF). One mechanism for renocardiac injury in AKI is activation of the renin-angiotensin system (RAS) with increased production of Angiotensin II (ANGII). Patients undergoing cardiovascular surgery are at especially high risk for AKI. There is a need to develop novel therapeutics for AKI that may reduce the risk for future HF and also target the RAS.

### SUMMARY

NPs are polypeptides that can cause natriuresis (increased sodium excretion in the urine). NPs include atrial natriuretic peptide (ANP), B-type natriuretic peptide (BNP), C-type natriuretic peptide (CNP), and urodilatin (URO). NPs can be produced by the brain, heart, kidney, and/or vascular tissue. NPs function via guanylyl cyclase receptors (NPR-A for ANP, BNP, and URO, and NPR-B for CNP) and the second messenger cyclic 3′5′ guanosine monophosphate (cGMP) (Kuhn, Circ Res, 93:700-709, 2003; Tawaragi et al., Biochem Biophys Res Commun, 175:645-651, 1991; and Komatsu et al., Endocrinol, 129:1104-1106, 1991).

CRRL269 is a “designer” NP that includes amino acid segments from ANP, BNP, and URO. This document is based, at least in part, on the discovery that CRRL269 is a renocardiac protective guanylyl cyclase A (GC-A) receptor activator that (1) increases renal tubular cell cGMP generation with less hypotension than native GC-A activators (e.g., BNP and URO), as demonstrated in a large animal model of AKI produced by aortic crossing clamping (ACC, a clinically relevant maneuver), and (2) potently suppresses RAS. In addition, this document is based, at least in part, on the discovery that urinary CNP (uCNP) is useful as a biomarker for AKI.

In a first aspect, therefore, this document features a method that includes measuring the level of CNP in a biological sample from a mammal, identifying the mammal as having a CNP level that is elevated related to a corresponding control level of CNP, and administering to the mammal a composition containing a peptide that includes, in order from N-terminus to C-terminus: (a) the sequence set forth in SEQ ID NO:1 with three or less amino acid additions, deletions, substitutions, or combinations thereof, (b) the sequence set forth in SEQ ID NO:2 with five or less amino acid additions, deletions, substitutions, or combinations thereof, and (c) the sequence set forth in SEQ ID NO:3 with two or less amino acid additions, deletions, substitutions, or combinations thereof. The mammal can be a human (e.g., a human who is undergoing or has undergone cardiovascular surgery). The biological sample can be a urine sample or a blood sample. The measuring can include determining a level of CNP mRNA in the biological sample or determining a level of CNP protein in the biological sample. In some cases, the measuring can include a radioimmunoassay. The method can include administering to the mammal a composition containing a peptide having the sequence of SEQ ID NO:4. The peptide can be administered at a dose of about 0.001 to 0.01 μg/kg/minute for 24 to 96 hours (e.g., a dose of about 0.005 μg/kg/minute for 48 to 72 hours). The method can further include administering a diuretic to the mammal.

In another aspect, this document features a method for identifying a mammal in need of treatment for AKI. The method can include measuring the level of CNP in a biological sample obtained from a mammal, and identifying the mammal as having an elevated level of CNP in the biological sample, as compared to a control level of CNP, thereby identifying the mammal as being in need of a therapy for AKI. The mammal can be a human (e.g., a human who is undergoing or has undergone cardiovascular surgery). The biological sample can be a urine sample or a blood sample. The measuring can include determining a level of CNP mRNA in the biological sample, or determining a level of CNP protein in the biological sample. In some cases, the measuring can include a radioimmunoassay. The therapy can include a peptide containing, in order from N-terminus to C-terminus: (a) the sequence set forth in SEQ ID NO:1 with three or less amino acid additions, deletions, substitutions, or combinations thereof, (b) the sequence set forth in SEQ ID NO:2 with five or less amino acid additions, deletions, substitutions, or combinations thereof, and (c) the sequence set forth in SEQ ID NO:3 with two or less amino acid additions, deletions, substitutions, or combinations thereof. The therapy can include a peptide having the amino acid sequence of SEQ ID NO:4.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic diagram of a CRRL269 polypeptide (SEQ ID NO:4). The first ten amino acid residues of CRRL269 correspond to amino acid residues 1 to 10 of human urodilatin (TAPRSLRRSS; SEQ ID NO:1). Amino acid residues 11 to 27 of CRRL269 correspond to amino acid residues 10 to 26 of human mature BNP (CFGRKMDRISSSSGLGC; SEQ ID NO:2). Amino acid residues 28 to 32 of CRRL269 correspond to amino acid residues 24 to 28 of human ANP (NSFRY; SEQ ID NO:3).

FIGS. 2A-2D show that plasma cGMP, urinary cGMP, renal cGMP and plasma angiotensin II (ANGII) values in AKI canines were modulated by CRRL269. Specifically, FIG. 2A is a graph plotting plasma cGMP, FIG. 2B is a graph plotting urinary cGMP, FIG. 2C is a graph plotting renal cortical and medullary cGMP, and FIG. 2D is a graph plotting plasma ANGII values in AKI canines after administration of CRRL269 or vehicle. The AKI protocol consisted of baseline (BL), indomethacin (INDO) infusion, aortic cross clamping (ACC), and clearances 1 through 4 (CL1, CL2, CL3 and CL4). After a 60 minute equilibration, BL values were measured. A 45 minute infusion of INDO was initiated, followed by 60 minutes of ACC. Infusion of 16.3 pmol/kg/min CRRL269 or vehicle (0.9% saline) started after ACC and lasted for 120 minutes, with clearance samples being taken at 30, 60, 90, and 120 minutes. Data were recorded at BL, INDO, ACC, CL1, CL2, CL3 and CL4 during CRRL269/vehicle infusion. * p<0.05 vs baseline. #p<0.05 vs vehicle.

FIGS. 3A-3D provide data for renal function parameters in AKI canines after CRRL269 treatment. FIG. 3A is a graph plotting glomerular filtration rate (GFR), FIG. 3B is a graph plotting renal blood flow (RBF), FIG. 3C is a graph plotting urine output (UV), and FIG. 3D is a graph plotting urinary sodium excretion rate (UNaV) in CRRL269 and vehicle infusion groups. Data are presented as absolute changes from baseline. The experimental schedule was as described for FIGS. 2A-2D. * p<0.05 vs baseline, #p<0.05 vs vehicle.

FIGS. 4A-4D provide data for cardiovascular function parameters in AKI canines after CRRL269 treatment. FIG. 4A is a graph plotting mean arterial pressure (MAP), FIG. 4B is a graph plotting cardiac output (CO), FIG. 4C is a graph plotting right atrial pressure (RAP), and FIG. 4D is a graph plotting pulmonary capillary wedge pressure (PCWP) in CRRL269 and vehicle infusion groups. Data are presented as absolute changes from baseline. The experimental schedule was as described for FIGS. 2A-2D. * p<0.05 vs baseline.

FIGS. 5A-5D show the results of H&E staining and ex vivo TUNEL apoptosis studies in AKI canine kidneys. FIG. 5A includes representative images of H&E staining of kidney cortex in vehicle and CRRL269 groups, as indicated. Arrow heads indicate vacuolization or tubular dilation. FIG. 5B is a graph plotting renal cortex vacuolization scores. FIG. 5C includes representative images showing kidney cortex apoptosis assessed by TUNEL assay in normal, vehicle, and CRRL269 groups, analyzed by con-focal fluorescence microscopy. Green fluorescence indicates DNA fragmentation and blue fluorescence labels the nucleus. FIG. 5D is a graph plotting quantified TUNEL results. * p<0.05 vs normal, #p<0.05 vs vehicle.

FIGS. 6A-6E show that CRRL269 inhibits apoptosis induced by hypoxia/reoxygenation (H/R) in human primary renal proximal tubular epithelial cells (HRPTC). FIG. 6A is a graph plotting apoptotic signals quantified by IncuCyte software for cells treated by H/R or H/R with three concentrations (10⁻⁸, 10⁻⁷, or 10⁻⁶ M) of ANP, BNP, or CRRL269. FIG. 6B is a graph plotting apoptotic signals in HRPTC treated by H/R or H/R with three concentrations (10⁻⁸, 10⁻⁷, or 10⁻⁶ M) of the protein kinase G- (PKG-) specific cGMP analogue, 8-pCPTcGMP, which has low affinity for phosphodiesterases. FIG. 6C is a graph plotting apoptosis for HRPTC treated by H/R or H/R with four concentrations (0, 1, 5, or 15 μM) of the PKG inhibitor KT-5823 (KT) with 10⁻⁶ M CRRL269. FIG. 6D is a pair of images showing apoptosis pathway gene expression compared between H/R and negative control (left panel), or H/R and 10⁻⁶ M CRRL269 (right panel). FIG. 6E is a graph plotting the level of Caspase7 gene expression for negative control, H/R, and H/R with 10⁻⁶ M CRRL269, as determined by RT-PCR. * p<0.05 vs negative control, #p<0.05 vs H/R.

FIGS. 7A and 7B show intracellular Ca2⁺ levels in human primary aortic vascular smooth muscle cells (HASMC). FIG. 7A is a graph plotting relative Ca2⁺ levels, which were calculated based on recorded fluorescence signals in Fura-2 loaded HASMC cells. Fold difference F/FO was used as relative Ca2⁺ concentration values. Cells were pretreated with 10⁻⁹ M ANP, BNP, or CRRL269 for 5 minutes before 10⁻⁸ M ANGII was perfused. Perfusion lasted for about 4 minutes. FIG. 7B is a graph plotting relative Ca2⁺ in HASMC cells that were pretreated with 10⁻⁷ M ANP, BNP, or CRRL269 for 5 minutes before 10⁻⁷ M ANGII was perfused.

FIGS. 8A-8C show CNP changes induced by CRRL269 in canine AKI. FIG. 8A is a graph plotting plasma CNP levels, and FIG. 8B is a graph plotting urinary CNP levels in canine AKI studies as described for FIGS. 2A-2D. FIG. 8C is a graph plotting renal CNP (NPPC) mRNA levels determined by RT-PCR, with normalization to GAPDH for CRRL269 or vehicle infusion groups. Data are presented as absolute changes from baseline. * p<0.05 vs baseline, #p<0.05 vs vehicle.

FIGS. 9A and 9B show in vitro CNP gene expression and renal fibroblast proliferation after CNP treatment. FIG. 9A is a graph plotting CNP gene (NPPC) mRNA levels as determined by RT-PCR in human glomerular microvascular endothelial cells (HGMEC) and human renal proximal tubular epithelial cells (HPRTC). FIG. 9B is a graph plotting proliferation of human renal fibroblasts (HRF), assessed by classical cell counting. HRF were treated with PBS or two concentrations (10⁻⁸ or 10⁻⁷ M) of CNP every 24 hours, and cell numbers were counted at 72 hours.

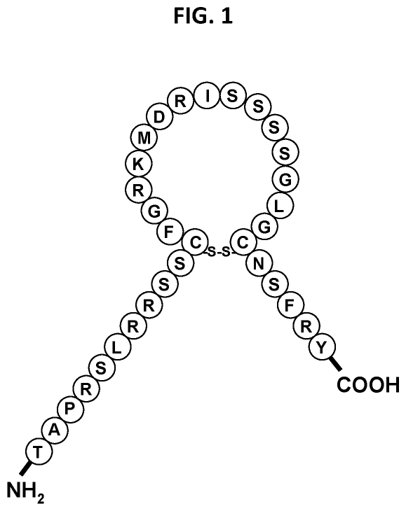

### DETAILED DESCRIPTION

This document relates to methods for using NPs (e.g., CNP) to identify mammals as having (or being likely to have) AKI and therefore being in need of treatment for AKI, as well as methods of using NPs (e.g., CPPL269) to treat mammals identified as being in need thereof. For example, this document provides methods and materials related to identifying mammals as having (or being likely to have) AKI based on the mammal's urinary CNP level, and methods and materials related to treating mammals identified as having (or being likely to have) AKI with CRRL269.

Thus, in some embodiments, this document provides methods that include detecting and/or measuring the level of a NP such as CNP. CNP is expressed as a 126 amino acid precursor that is cleaved to the 22 amino acid mature CNP. The amino acid sequence of the human preproprotein is MHLSQLLACALLLTLLSLRPSEAK PGAPPKVPRTPPAEELAEPQAAGGGQKKGDKAPGGGGANLKGDRSRLLRDL RVDTKSRAAWARLLQEHPNARKYKGANKKGLSKGCFGLKLDRIGSMSGLGC (SEQ ID NO:5), and the amino acid sequence of mature human CNP is GLSKGCF GLKLDRIGSMSGLGC (SEQ ID NO:6). A representative nucleotide sequence encoding SEQ ID NO:5 is: ATGCATCTCTCCCAGCTGCTGGCCTGCGCCCTGC TGCTCACGCTGCTCTCCCTCCGGCCCTCCGAAGCCAAGCCCGGGGCGCCG CCGAAGGTCCCGCGAACCCCGCCGGCAGAGGAGCTGGCCGAGCCGCAGG CTGCGGGCGGCGGTCAGAAGAAGGGCGACAAGGCTCCCGGGGGCGGGGG CGCCAATCTCAAGGGCGACCGGTCGCGACTGCTCCGGGACCTGCGCGTGG ACACCAAGTCGCGGGCAGCGTGGGCTCGCCTCTGCAAGAGCACCCCAAC GCGCGCAAATACAAAGGAGCCAACAAGAAGGGCTGTCCAAGGGCTGCT CGGCCTCAAGCTGGACCGAATCGGCTCCATGAGCGGCCTGGGATGTAGT (SEQ ID NO:7). A representative nucleotide sequence encoding SEQ ID NO:6 is:

(SEQ ID NO: 8)
GGCTTGTCCAAGGGCTGCTTCGGCCTCAAGCTGG
ACCGAATCGGCTCCATGAGCGGCCTGGGATGT.

The methods provided herein can be used to measure CNP levels in a biological sample from a subject (e.g., a mammal, such as a human, non-human primate, mouse, rat, rabbit, pig, sheep, dog, cat, or horse), and identifying the subject as having (or being likely to have) AKI when the sample contains an elevated level of CNP, relative to a corresponding control level of CNP. In some cases, the subject can be undergoing, or can have undergone, cardiovascular surgery. In some cases, the level of CNP in a sample obtained from a subject can indicate the severity of AKI. For example, higher levels of CNP can be associated with more severe cases of AKI. Suitable biological samples include fluid samples such as, without limitation, urine, blood, serum, plasma, or cerebrospinal fluid.

Any appropriate method can be used to detect and/or quantify the level of a NP (e.g., CNP), including, for example, immunological methods that utilize one or more antibodies. As used herein, the term “antibody” includes intact molecules as well as fragments thereof that are capable of binding to an epitopic determinant of a NP. The term “epitope” refers to an antigenic determinant on an antigen to which the paratope of an antibody binds. Epitopic determinants usually consist of chemically active surface groupings of molecules such as amino acids or sugar side chains, and typically have specific three-dimensional structural characteristics, as well as specific charge characteristics. Epitopes generally have at least five contiguous amino acids (a continuous epitope), or alternatively can be a set of noncontiguous amino acids that define a particular structure (e.g., a conformational epitope). The term “antibody” includes polyclonal antibodies, monoclonal antibodies, humanized or chimeric antibodies, single chain Fv antibody fragments, Fab fragments, and F(ab)₂ fragments. Polyclonal antibodies are heterogenous populations of antibody molecules that are contained in the sera of the immunized animals. Monoclonal antibodies are homogeneous populations of antibodies to a particular epitope of an antigen.

Antibody fragments that have specific binding affinity for a NP (e.g., CNP) can be generated by known techniques. For example, F(ab′)2 fragments can be produced by pepsin digestion of the antibody molecule; Fab fragments can be generated by reducing the disulfide bridges of F(ab′)2 fragments. In some cases, Fab expression libraries can be constructed. See, for example, Huse et al., Science, 246:1275 (1989). Once produced, antibodies or fragments thereof can be tested for recognition of a particular target (e.g., CNP) by standard immunoassay methods such as ELISA techniques, radioimmunoassays, and Western blotting. See, Short Protocols in Molecular Biology, Chapter 11, Green Publishing Associates and John Wiley & Sons, Ed. Ausubel et al., 1992.

In immunological assays, an antibody having specific binding affinity for a polypeptide provided herein or a secondary antibody that binds to such an antibody can be labeled, either directly or indirectly. Suitable labels include, without limitation, radionuclides (e.g., ¹²⁵I, ¹³¹I, ³⁵S, ³H, ³²P, ³³P, or ¹⁴C), fluorescent moieties (e.g., fluorescein, FITC, PerCP, rhodamine, or PE), luminescent moieties (e.g., Qdot™ nanoparticles supplied by Invitrogen; Carlsbad, Calif.), compounds that absorb light of a defined wavelength, or enzymes (e.g., alkaline phosphatase or horseradish peroxidase). Antibodies can be indirectly labeled by conjugation with biotin then detected with avidin or streptavidin labeled with a molecule described above. Methods of detecting or quantifying a label depend on the nature of the label and are known in the art. Examples of detectors include, without limitation, x-ray film, radioactivity counters, scintillation counters, spectrophotometers, colorimeters, fluorometers, luminometers, and densitometers. Combinations of these approaches (including “multi-layer” assays) familiar to those in the art can be used to enhance the sensitivity of assays.

Immunological assays for detecting a NP (e.g., CNP) can be performed in a variety of known formats, including sandwich assays, competition assays (competitive RIA), or bridge immunoassays. See, for example, U.S. Pat. Nos. 5,296,347; 4,233,402; 4,098,876; and 4,034,074. Methods of detecting a NP generally can include contacting a biological sample with an antibody that binds to the NP and detecting binding of the polypeptide to the antibody. For example, an antibody having specific binding affinity for CNP can be immobilized on a solid substrate by any appropriate method and then exposed to the biological sample. Binding of the CNP to the antibody on the solid substrate can be detected by exploiting the phenomenon of surface plasmon resonance, which results in a change in the intensity of surface plasmon resonance upon binding that can be detected qualitatively or quantitatively by an appropriate instrument, e.g., a Biacore apparatus (Biacore International AB; Rapsgatan, Sweden). In some cases, the antibody can be labeled and detected as described above. A standard curve using known quantities of a NP (e.g., CNP) can be generated to aid in the quantitation of the levels of the polypeptide.

In some embodiments, a “sandwich” assay in which a capture antibody is immobilized on a solid substrate can be used to detect the presence, absence, or level of a NP (e.g., CNP). The solid substrate can be contacted with the biological sample such that the polypeptide of interest in the sample can bind to the immobilized antibody. The presence, absence, or level of the polypeptide bound to the antibody can be determined using a “detection” antibody having specific binding affinity for the polypeptide. In some embodiments, a capture antibody can be used that has binding affinity for ANP, BNP, or urodilatin as well as CNP. In such embodiments, a detection antibody can be used that has specific binding affinity for CNP. It is understood that in sandwich assays, the capture antibody should not bind to the same epitope (or range of epitopes in the case of a polyclonal antibody) as the detection antibody. Thus, if a monoclonal antibody is used as a capture antibody, the detection antibody can be another monoclonal antibody that binds to an epitope that is either physically separated from or only partially overlaps with the epitope to which the capture monoclonal antibody binds, or a polyclonal antibody that binds to epitopes other than or in addition to that to which the capture monoclonal antibody binds. If a polyclonal antibody is used as a capture antibody, the detection antibody can be either a monoclonal antibody that binds to an epitope that is either physically separated from or partially overlaps with any of the epitopes to which the capture polyclonal antibody binds, or a polyclonal antibody that binds to epitopes other than or in addition to that to which the capture polyclonal antibody binds. Sandwich assays can be performed as sandwich ELISA assays, sandwich Western blotting assays, or sandwich immunomagnetic detection assays.

Suitable solid substrates to which an antibody (e.g., a capture antibody) can be bound include, without limitation, microtiter plates, tubes, membranes such as nylon or nitrocellulose membranes, and beads or particles (e.g., agarose, cellulose, glass, polystyrene, polyacrylamide, magnetic, or magnetizable beads or particles). Magnetic or magnetizable particles can be particularly useful when an automated immunoassay system is used.

Antibodies having specific binding affinity for a NP (e.g., CNP) can be produced through standard methods. For example, a polypeptide can be recombinantly produced as described above, can be purified from a biological sample (e.g., a heterologous expression system), or can be chemically synthesized, and used to immunize host animals, including rabbits, chickens, mice, guinea pigs, or rats. For example, a polypeptide having the amino acid sequence set forth in SEQ ID NO:5 or SEQ ID NO:6, or fragments thereof that are at least six amino acids in length, can be used to immunize an animal. Various adjuvants that can be used to increase the immunological response depend on the host species and include Freund's adjuvant (complete and incomplete), mineral gels such as aluminum hydroxide, surface active substances such as lysolecithin, pluronic polyols, polyanions, peptides, oil emulsions, keyhole limpet hemocyanin and dinitrophenol. Monoclonal antibodies can be prepared using a polypeptide provided herein and standard hybridoma technology. In particular, monoclonal antibodies can be obtained by any technique that provides for the production of antibody molecules by continuous cell lines in culture such as described by Kohler et al., Nature, 256:495 (1975), the human B-cell hybridoma technique (Kosbor et al., Immunology Today, 4:72 (1983); Cole et al., Proc. Natl. Acad. Sci. USA, 80:2026 (1983)), and the EBV-hybridoma technique (Cole et al., “Monoclonal Antibodies and Cancer Therapy,” Alan R. Liss, Inc., pp. 77-96 (1983)). Such antibodies can be of any immunoglobulin class including IgG, IgM, IgE, IgA, IgD, and any subclass thereof. The hybridoma producing the monoclonal antibodies can be cultivated in vitro and in vivo.

Other suitable techniques for detecting and/or quantifying the level of a NP (e.g., CNP) in a sample can include mass-spectrophotometric techniques such as electrospray ionization (ESI), and matrix-assisted laser desorption-ionization (MALDI). See, for example, Gevaert et al., Electrophoresis, 22(9):1645-51 (2001); Chaurand et al., J. Am. Soc. Mass Spectrom., 10(2):91-103 (1999). Mass spectrometers useful for such applications are available from Applied Biosystems (Foster City, Calif.); Bruker Daltronics (Billerica, Mass.); and Amersham Pharmacia (Sunnyvale, Calif.). In addition, methods for detecting and/or quantifying the level of a NP (e.g., CNP) can include nucleic acid-based techniques such as Northern blotting, nuclease protection, in situ hybridization, and PCR-based methods (e.g., reverse transcription-polymerase chain reaction; RT-PCR) to detect and quantify the NP mRNA.

The methods provided herein, in some embodiments, can include identifying a subject (e.g., a mammal such as a human) as having an increased level of CNP (e.g., CNP protein or CNP mRNA) relative to a corresponding control. In some cases, the subject can be a patient who is undergoing or has undergone cardiovascular surgery. Subjects who are identified according to any of these criteria can be classified as having, or being likely to have, AKI. Conversely, subjects who are identified as not having CNP levels, relative to a corresponding control, can be classified as =having or being likely to have AKI.

The term “increased” as used herein with respect to CNP levels refers to a level that is greater (e.g., at least 5% greater, at least 10% greater, at least 25% greater, at least 50% greater, 5 to 10% greater, 10 to 25% greater, 25 to 50% greater, 50 to 75% greater, at least 2-fold greater, at least 3-fold greater, at least 5-fold greater, 2- to 3-fold greater, or 3- to 5-fold greater) than a “reference” or “control” level of CNP. The terms “reference” or “control,” as used herein with respect to CNP levels, can refer to the level of CNP protein or mRNA in a corresponding subject or population of subjects that is known not to have AKI. In some cases, a control level of CNP can be a level measured at baseline (e.g., prior to cardiovascular surgery).

Thus, in some embodiments, this document provides methods that include measuring the level of CNP in a biological sample (e.g., blood or urine) obtained from a subject (e.g., a mammal such as a human who is undergoing or has undergone cardiovascular surgery), and identifying the subject as having or being likely to have AKI when the level of CNP in the biological sample is increased relative to a corresponding control level of CNP. The increase in CNP expression can be relative to a corresponding control level, such as the level in a corresponding subject that does not have AKI. The identification of a mammal as having, or being likely to have, AKI also can indicate that the mammal is likely to respond to treatment with CRRL269. In some cases, therefore, the methods also can include treating the identified subject with CRRL269.

Having the ability to identify mammals as having AKI and as being likely to respond to a certain treatment (e.g., treatment with CRRL269, or a pharmaceutical composition containing CRRL269) can allow those mammals to be properly identified and treated in an effective and reliable manner. For example, the treatments described herein (e.g., CRRL269) can be used to treat patients identified as having (or being likely to have) AKI based on their CNP expression.

CRRL269 is a peptide that includes amino acid segments from, or based on, ANP, BNP, and URO. The CRRL269 polypeptide used in the methods of treatment provided herein can include (e.g., in order from N-terminus to C-terminus) the amino acid sequences set forth in SEQ ID NO:1, SEQ ID NO:2, and SEQ ID NO:3. In some cases, a CRRL269 polypeptide provided herein can contain an amino acid sequence that aligns to (a) the sequence set forth in SEQ ID NO:1 with three or less (e.g., two or less, one, or zero) amino acid additions, deletions, substitutions, or combinations thereof, (b) the sequence set forth in SEQ ID NO:2 with five or less (e.g., four or less, three or less, two or less, one, or zero) amino acid additions, deletions, substitutions, or combinations thereof and (c) the sequence set forth in SEQ ID NO:3 with two or less (e.g., one or zero) amino acid additions, deletions, substitutions, or combinations thereof. For example, a CRRL269 polypeptide can contain the sequence set forth in SEQ ID NO:1, with the exception that the first occurring proline residue or the last occurring serine residue of SEQ ID NO:1 is deleted or replaced with a different amino acid residue. In some cases, a polypeptide provided herein can contain an amino acid sequence that aligns to (a) the sequence set forth in SEQ ID NO:1 with three or less (e.g., two or less, one, or zero) amino acid additions, deletions, substitutions, or combinations thereof, provided that the polypeptide contains the TAPR amino acid sequence, (b) the sequence set forth in SEQ ID NO:2 with five or less (e.g., four or less, three or less, two or less, one, or zero) amino acid additions, deletions, substitutions, or combinations thereof, and (c) the sequence set forth in SEQ ID NO:3 with two or less (e.g., one or zero) amino acid additions, deletions, substitutions, or combinations thereof. In some cases, the presence of an N-terminal TAPR amino acid sequence can enhance the cGMP-activating and renal actions of the polypeptide. In some cases, a CRRL269 polypeptide can comprise, consist essentially of, or consist of, the sequence set forth in SEQ ID NO:4.

Amino acid substitutions can be conservative amino acid substitutions. Conservative amino acid substitutions can be, for example, aspartic acid/glutamic acid as acidic amino acids; lysine/arginine/histidine as basic amino acids; leucine/isoleucine, methionine/valine, alanine/valine as hydrophobic amino acids; and serine/glycine/alanine/threonine as hydrophilic amino acids. Conservative amino acid substitutions also include groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. After making an amino acid substitution, the activities of the polypeptide containing the amino acid substitution can be assessed using the assays described herein.

A CRRL269 polypeptide can have a length between 22 and 42 (e.g., between 22 and 42, between 24 and 40, between 25 and 39, between 26 and 38, between 27 and 37, between 28 and 36, between 29 and 35, between 30 and 34, between 25 and 30, or between 30 and 35) amino acid residues in length. It will be appreciated that a polypeptide with a length of 22 or 42 amino acid residues is a polypeptide with a length between 22 and 42 amino acid residues.

In some cases, a CRRL269 polypeptide can be substantially pure. As used herein, the term “substantially pure” with reference to a polypeptide means that the polypeptide is substantially free of other polypeptides, lipids, carbohydrates, and nucleic acid with which it is naturally associated. Thus, a substantially pure polypeptide is any polypeptide that is removed from its natural environment and is at least 60 percent pure or is any chemically synthesized polypeptide. A substantially pure polypeptide can be at least about 60, 65, 70, 75, 80, 85, 90, 95, or 99 percent pure. Typically, a substantially pure polypeptide will yield a single major band on a non-reducing polyacrylamide gel.

A CRRL269 polypeptide can be obtained by expression of a recombinant nucleic acid encoding the polypeptide or by chemical synthesis (e.g., using solid phase polypeptide synthesis methods or an peptide synthesizer such as an ABI 431A Peptide Synthesizer; Applied Biosystems; Foster City, Calif.). In some cases, for example, standard recombinant technology using an expression vector encoding a CRRL269 polypeptide can be used. The resulting polypeptide then can be purified using, for example, affinity chromatographic techniques and HPLC. The extent of purification can be measured by any appropriate method, including but not limited to: column chromatography, polyacrylamide gel electrophoresis, or high-performance liquid chromatography. A CRRL269 polypeptide can be designed or engineered to contain a tag sequence that allows the polypeptide to be purified (e.g., captured onto an affinity matrix). For example, a tag such as c-myc, hemagglutinin, polyhistidine, or FLAG™ tag (Kodak) can be used to aid polypeptide purification. Such tags can be inserted anywhere within the polypeptide, including at either the carboxyl or amino terminus. Other fusions that can be used include enzymes that aid in the detection of the polypeptide, such as alkaline phosphatase.

A CRRL269 polypeptide can be used to treat AKI. For example, a CRRL269 polypeptide as described herein (e.g., having the amino acid sequence set forth in SEQ ID NO:4) can be administered to a mammal (e.g., a human) identified as having, or being likely to have, AKI, where the administering is under conditions wherein the severity of the mammal's AKI symptoms is reduced after the administering.

Thus, a CRRL269 polypeptide can be formulated as a pharmaceutical composition by admixture with one or more pharmaceutically acceptable non-toxic excipients or carriers. Such compositions can be administered to a mammal in need thereof in an amount effective to treat AKI. Pharmaceutical compositions can be prepared for parenteral administration, particularly in the form of liquid solutions or suspensions in aqueous physiological buffer solutions; for oral administration, particularly in the form of tablets or capsules; or for intranasal administration, particularly in the form of powders, nasal drops, or aerosols. Compositions for other routes of administration can be prepared as desired using appropriate methods.

Formulations for parenteral administration can include as common excipients, sterile water, saline, polyalkylene glycols such as polyethylene glycol, oils of vegetable origin, hydrogenated naphthalenes, and combinations thereof. In some cases, biocompatible, biodegradable lactide polymer, lactide/glycolide copolymer, polyoxethylene-polyoxypropylene copolymers, or combinations thereof can be used as excipients for controlling the release of the polypeptide in vivo. Other suitable parenteral delivery systems that can be used include, without limitation, ethylene-vinyl acetate copolymer particles, osmotic pumps, implantable infusion systems, liposomes, and combinations thereof. Formulations for inhalation administration can include excipients such as lactose. Inhalation formulations can be aqueous solutions containing, for example, polyoxyethylene-9-lauryl ether, glycocholate, deoxycholate, or combinations thereof, or they can be oily solutions for administration in the form of nasal drops. If desired, a composition containing a polypeptide provided herein can be formulated as gel to be applied intranasally. Formulations for parenteral administration can include glycocholate for buccal administration.

For oral administration, tablets or capsules can be prepared using appropriate methods with pharmaceutically acceptable excipients such as binding agents (e.g., pregelatinized maize starch, polyvinylpyrrolidone, or hydroxypropyl methylcellulose); fillers (e.g., lactose, microcrystalline cellulose or calcium hydrogen phosphate); lubricants (e.g. magnesium stearate, talc or silica); disintegrants (e.g., potato starch or sodium starch glycolate); or wetting agents (e.g., sodium lauryl sulfate). Tablets can be coated using appropriate methods. Preparations for oral administration can be formulated to give controlled release of the polypeptide.

Nasal preparations can be presented in a liquid form or as a dry product. Nebulised aqueous suspensions or solutions can include carriers or excipients to adjust pH and/or tonicity.

Suitable methods for administering polypeptides such as CRRL269 include, for example, parenteral administration (e.g., by subcutaneous, intrathecal, intraventricular, intramuscular, or intraperitoneal injection, or by intravenous drip). Administration can be rapid (e.g., by injection) or can occur over a period of time (e.g., by slow infusion or administration of slow release formulations). In some embodiments, administration can be topical (e.g., transdermal, sublingual, ophthalmic, or intranasal), pulmonary (e.g., by inhalation or insufflation of powders or aerosols), or oral. In addition, a therapy can be administered prior to, after, or in lieu of surgical resection of a tumor.

CRRL269 can be administered to a subject in an appropriate amount, at an appropriate frequency, and for an appropriate duration effective to achieve a desired outcome (e.g., a decrease in symptoms of AKI). Symptoms of AKI can include one or more of the following: water-electrolyte imbalance, fatigue, insufficient urine production, urine retention, shortness of breath, swelling, too much acid in the blood, elevated serum creatinine and blood urea nitrogen (BUN), and decreased glomerular filtration rate; structural changes in the kidney symptomatic of AKI include acute tubular injury (e.g., tubular apoptosis and necrosis with marked renal vasoconstriction). In some cases, CRRL269 can be administered to a subject identified as having AKI to reduce one or more symptoms of AKI by at least 5 percent (e.g., at least 5 percent, at least 10 percent, at least 25 percent, at least 50 percent, at least 75 percent, or 100 percent). For example, the progression rate can be reduced such that no further AKI symptoms are detected. Any appropriate method can be used to determine whether or not a subject is experiencing symptoms of AKI.

An effective amount of a composition containing CRRL269 can be any amount that reduces a symptom of AKI without producing significant toxicity to the mammal. Optimum dosages can vary depending on the relative potency of the peptide, and can generally be estimated based on EC₅₀ found to be effective in in vitro and in vivo animal models. Typically, dosage is from 5 μg to 100 μg per kg of body weight. For example, an effective amount of CRRL269 can be from about 5 to 10 μg/kg, about 7 to 15 μg/kg, about 10 to 20 μg/kg, about 14 to 22 μg/kg, about 20 to 50 μg/kg, about 22 to 29 μg/kg, about 50 to 75 μg/kg, or about 75 to 100 μg/kg. If a particular subject fails to respond to a particular amount, then the amount of the therapy can be increased by, for example, two-fold. After receiving this higher concentration, the subject can be monitored for both responsiveness to the treatment and toxicity symptoms, and adjustments made accordingly. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the cancer may require an increase or decrease in the actual effective amount administered.

The CRRL269 can be administered once or more than once, at any appropriate frequency and for any appropriate duration. For example, the frequency of administration can be once or more daily, biweekly, weekly, monthly, or even less. The frequency of administration can remain constant or can be variable during the duration of treatment. A course of treatment can include rest periods. For example, a composition containing CRRL269 can be administered over a two week period followed by a two week rest period, and then repeated. As with the effective amount, various factors can influence the actual frequency of administration. For example, the effective amount, duration of treatment, use of multiple treatment agents, route of administration, and severity of the AKI symptoms may require an increase or decrease in administration frequency.

An effective duration for administering a therapy such as CRRL269 can be any duration that reduces AKI symptoms without producing significant toxicity to the subject. Thus, the effective duration can vary from several hours to several days, or even weeks or months. In general, the effective duration for the treatment of AKI can range from about 24 to 96 hours (e.g., about 24 to 48 hours, about 48 to 72 hours, or about 72 to 96 hours), although in some cases, an effective duration can be for as long as an individual subject is alive. In some cases, a composition containing CRRL269 can be administered at a dose of about 0.001 to about 0.010 μg/kg/min for 24 to 96 hours (e.g., about 0.005 μg/kg/min for 48 to 72 hours). Multiple factors can influence the actual effective duration used for a particular treatment. For example, an effective duration can vary with the frequency of administration, effective amount, use of multiple treatment agents, route of administration, and severity of the AKI.

In some embodiments, the CRRL269 can be administered with another therapeutic, such as a diuretic (e.g., furosemide, bumetanide, ethacrynic acid, torsemide, acetazolamide, dorzolamide, amiloride, spironolactone, eplerenone, triamterene, potassium canrenoate, bendroflumethiazide, or hydrochlorothiazide). The CRRL269 and the additional therapeutic can be administered simultaneously (e.g., in the same composition or in separate compositions that are administered at the same time), or sequentially.

After administering CRRL269 to a subject identified as having, or being like to have, AKI, the subject can be monitored to determine whether or not the AKI was treated. For example, a subject can be assessed after treatment to determine whether or not the AKI symptoms have been reduced (e.g., stopped). Any suitable method, including those that are known in the art, can be used to assess AKI severity.

The invention will be further described in the following examples, which do not limit the scope of the invention described in the claims.

### EXAMPLES

### Example 1—Materials and Methods

Ischemic Acute Kidney Injury in Canines: An ischemic AKI model in normal mongrel canines (male, 25-30 kg, n=5 per group) was created with high dose intravenous indomethacin infusion (10 mg/kg with NaHCO₃[5 mg/kg] dissolved in 50 ml distilled water) followed by suprarenal ACC (Sandok et al., J Am Soc Nephrol, 196-202, 1992). Briefly, canines were anesthetized with intravenous pentobarbital (15-30 mg/kg) and fentanyl (4-12 μg/kg), intubated, and mechanically ventilated on air with supplemental oxygen, 3 L/minute, 12 cycles/minute (Harvard Apparatus; Millis, Mass.). The femoral and carotid arteries were accessed for mean arterial pressure (MAP) monitoring and blood sampling. The femoral vein was cannulated for continuous inulin infusion and saline/indomethacin/drug infusions. The left renal artery was attached with a probe for renal blood flow (RBF) measurement and a catheter was placed in the left ureter for passive urine collection. A flow-directed balloon-tipped thermodilution catheter was advanced to the pulmonary artery through the external jugular vein for measurement of cardiac filling pressures and cardiac output (CO). After a 60 minute equilibration, baseline (BL) characteristics were established. A 45 minute infusion of indomethacin (INDO) was then started. A Blalock clamp was applied at the level of the suprarenal ACC to occlude blood flow to both kidneys. ACC was maintained for 60 minutes, and infusion of 16.3 pmol/kg/min CRRL269 or vehicle (0.9% saline) was initiated 5 minutes before releasing the clamp. Dogs were randomized to receive CRRL269 or vehicle infusion. Investigators were not blinded to treatments but biochemical analysis was performed by technicians who were blinded to treatment. The dose of CRRL269 was chosen based upon ANP dose used in previous AKI studies (Sandok et al., supra); this was a medium dose between low and high doses used in a study described elsewhere (Chen et al., Am J Physiol Regul Integr Comp Physiol, 314:R407-R414, 2018). Drug infusion lasted for 120 minutes, and data/samples were collected every 30 minutes, termed as Clearance 1 (CL1), Clearance 2 (CL2), Clearance 3 (CL3), and Clearance 4 (CL4). Immediately after completion of CL4, each animal was sacrificed, the left kidney was harvested and sliced, and slices were preserved in 10% formalin solution or snap frozen with liquid nitrogen. GFR values were calculated based on inulin clearance. Urinary sodium was measured with pHOx Ultra (Nova Biomedical; Waltham, Mass.).

Hormonal Measurements: Plasma and urinary cGMP were measured using commercially available ELISA kits (Enzo Life Sciences; Farmingdale, N.Y.). Ex vivo renal parenchyma cGMP levels were measured with the same ELISA kits. About 0.1 g frozen canine renal cortex or medulla (n=5 each, Veh or CRRL269) were homogenized in 2 mL 0.1N HCl with a homogenizer (Polytron PT1200E, Fisher Scientific; Waltham, Mass.). Homogenates were subjected to 600×g centrifugation for 10 minutes, and supernatants were collected for cGMP assay. Final cGMP levels were adjusted for protein concentration measured by the BSA method. Plasma ANGII was measured by radioimmunoassay (RIA) (Phoenix Pharmaceuticals; Burlingame, Calif.). Plasma and urinary CNP were measured by CNP RIA (Phoenix Pharmaceuticals). Both ELISA and RIA were performed as instructed by the manufacturers' protocols.

Histology and TUNEL Analysis: Fixed renal tissues were embedded in paraffin. For histology analysis, paraffin-embedded slides in vehicle (n=5) and CRRL269 (n=5) were stained with hematoxylin and eosin (H&E). A renal pathologist who was blinded to the study examined the H&E stained slides. Renal morphologic injuries such as tubular dilation and vacuolization were evaluated. Renal cortical apoptosis in vehicle (n=5) and CRRL269 (n=5) treated dogs were assessed using a TUNEL assay (Promega; Madison, Wis.) followed the protocol provided by the manufacturer. Normal canines (n=4, same background as the canines used in the current study) that did not undergo AKI or any other surgeries were added as an additional group to evaluate the renal apoptosis. DAPI (Vectashield, Vector Laboratories; Burlingame, Calif.) was used to label the nucleus. TUNEL results were analyzed by confocal fluorescence microscopy (Nikon A50R; Melville, N.Y.).

Oil Immersion Induced Hypoxia/Reoxygenation in vitro: HRPTC (ScienCell Research Laboratories; Carlsbad, Calif.) were seeded in 96-well plates and cultured for 1 day until 70-80% confluence. Passages 3-5 were used for experiments. Caspase 3/7 green apoptosis assay agent (Essen BioScience; Ann Arbor, Mich.) was added to the culture medium for detection of apoptosis. Plates were then transferred to a time-lapsed, live imaging system IncuCyte (Essen BioScience) for apoptosis monitoring for a total of 24 hours. H/R in cells was mimicked with mineral oil (Sigma, St Louis, Mo.) immersion (Meldrum et al., J Surg Res, 99:288-293, 2001). Cells were immersed in oil for 2 hours, washed twice, and placed in fresh medium containing vehicle (H/R group) or drugs. ANP, BNP, and CRRL269 were used at concentrations of 10⁻⁸, 10⁻⁷, or 10⁻⁶ M. PKG (an important cGMP binding protein) activating analogue 8-pCPT-cGMP (Sigma; St. Louis, Mo.), which is specific for PKG and has low affinity for phosphodiesterases, was used at concentrations of 10⁻⁶, 10⁻⁵, or 10⁻⁴ M. Cells were treated with 10⁻⁶-, 5×10⁻⁶-, or 1.5×10⁻⁵ M PKG inhibitor KT-5823 (Abcam; Cambridge, Mass.) for 30 minutes before 10⁻⁶ M CRRL269 was added. Cells without H/R stimulation were labeled as the negative control (Neg Ctrl) group. Apoptosis signals (e.g., apoptosis object counts) were analyzed with software provided by IncuCyte.

Apoptosis Pathway PCR Array and Caspase7 Real Time PCR: Two groups of HRPTC (vehicle and 10⁻⁶ M CRRL269) that underwent oil induced H/R, and a negative control (Neg Ctrl) group with no H/R stress, were cultured in 6-well plates as described above, and total RNA was collected using the Trizol method (Thermo Fisher Scientific; Waltham, Mass.). RNA was reverse transcribed for cDNA for PCR RT2 array experiments. Apoptosis pathway PCR array (Qiagen; Germantown, Md.) was conducted using the SYBR green (Bio-Rad Laboratories; Hercules, Calif.) method in a Lightcycler 480 (Roche Diagonostics; Indianapolis, Ind.). Raw data were analyzed with the online Qiagen RT2 PCR analysis program. The Caspase7 gene was up-regulated by H/R and down-regulated by CRRL269, both at least two-fold, and was selected for RTPCR verification. A Caspase7 RT-PCR primer (PPH00110C-200, Qiagen) was obtained, and RT-PCR reactions were performed using the SYBR green method in a Lightcycler 480. The B2M gene (primer PPH01094E, Qiagen) was used for normalization.

Measurements of Intracellular Calcium in Vascular Smooth Muscle Cells: HASMC (ScienCell Research Laboratories; Carlsbad, Calif.) were cultured and maintained as instructed by the manufacturer. Cells at passage 4 or 5 were used for experiments. Calcium imaging was performed as described elsewhere (Chai et al., Cardiovasc Res, 100:151-159, 2013). Briefly, cells were seeded on a 25 mm round cover glass (Fisher Scientific; Pittsburgh, Pa.) at a density of 5×10⁴ cells per slide. The slides had been pretreated with 0.5 N NaOH for 8 hours, rinsed with water, and sterilized before use. After overnight culturing, the slides with cells were assembled in a MS-02 Chamber (ALA Scientific Instrument; Farmingdale, N.Y.) and loaded with 3 μM fura-2 AM (Invitrogen; Carlsbad, Calif.) for 30 minutes in a 37° C. incubator. The chamber was then placed on the stage of an inverted Olympus IX71 microscope (Olympus America Inc.; Waltham, Mass.). The cells were maintained in 1 mL HASMC medium and treated with or without ANP, BNP, or CRRL269 at concentration of 10⁻⁹ or 10⁻⁷ M for 5 minutes before perfused with 5 mL medium containing ANGII (10⁻⁸ or 10⁻⁷ M). Intracellular Ca2⁺ fluorescence signals were measured as a ratio of fluorescence intensities at 510 nm from excitations of 340/380 nm, using a Hamamatsu ORCA-R2 CCD camera with a Sutter LB-LS/17 light source. Baseline fluorescence was recorded after 5 minutes of treatment, and fluorescence images were acquired continuously during the ANGII perfusion. Background fluorescence was subtracted and the calcium signal (F) was normalized to baseline fluorescence (FO) and expressed as a ratio (F/FO) using MetaFluor software (Olympus America Inc.).

NPPC mRNA Expression Analysis: For tissue CNP gene NPPC mRNA analysis, total RNA from 0.1 g canine cortex or medulla (n=5 for each group) was collected using the Trizol method. Total RNA from HGMEC (Cell Systems; Kirkland, Mass.) and HRPTC was collected using RNeasy kits (Qiagen). RT-PCR was performed using the SYBR method. The GAPDH gene was used for normalization for NPPC analysis. NPPC (QT00211946, Qiagen) and GAPDH (Hs99999905_ml, Thermo Fisher Scientific) primers were used.

Human Renal Fibroblasts Proliferation by Cell Counting: Human renal fibroblasts (HRF, Cell Biologics; Chicago, Ill.) were counted and seeded into 48-well plates with complete medium, and then incubated at 37° C., 5% CO₂ overnight. The culture medium was replaced with fresh medium containing PBS or CNP at concentrations of 10⁻⁸ or 10⁻⁷ M, and cells were cultured for 72 hours. Fresh CNP was added at 24 and 48 hours. At 72 hours, HRF were trypsinized, stained with 0.2% trypan blue, and counted using a Cedex XS Analyzer (Roche; Indianapolis, Ind.).

Statistics and Data Analysis: Data were expressed as mean f SEM and significance analysis was performed using Prism 7 (Graphpad; La Jolla, La.). Comparisons within a group were made by one way ANOVA, and two way ANOVA was used to compare the main effects of CRRL269 and vehicle in the in vivo and in vitro studies in which multiple time points were involved. Unpaired t-tests were performed for statistical analysis for in vitro and ex vivo studies with only a single time point.

### Example 2—Ischemic AKI Renal and Hormonal Function In Vivo in Canines

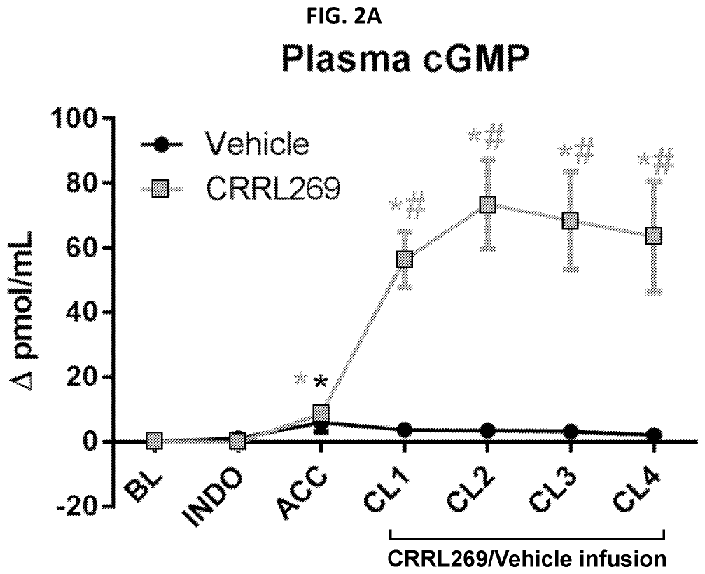

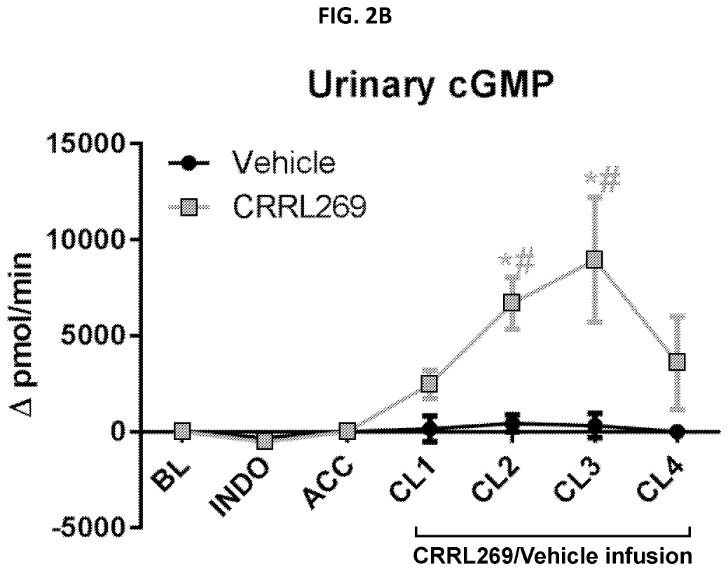

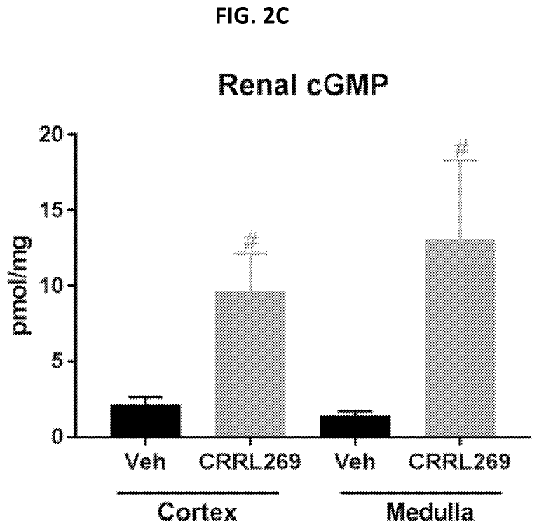

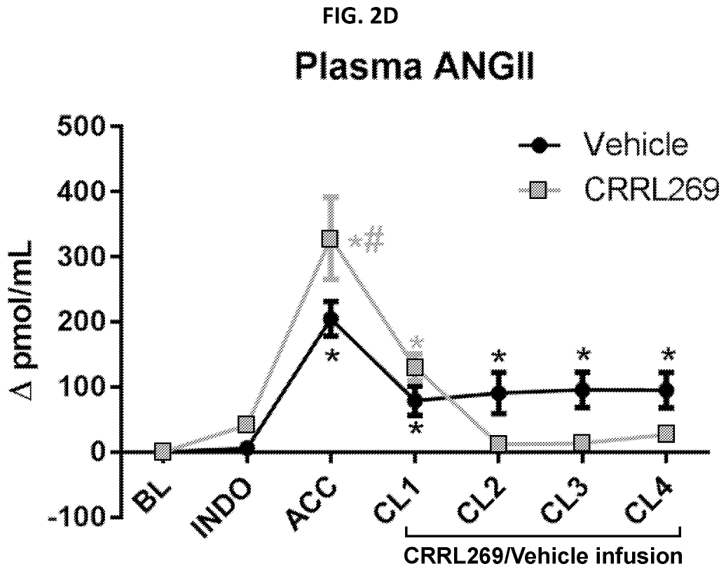

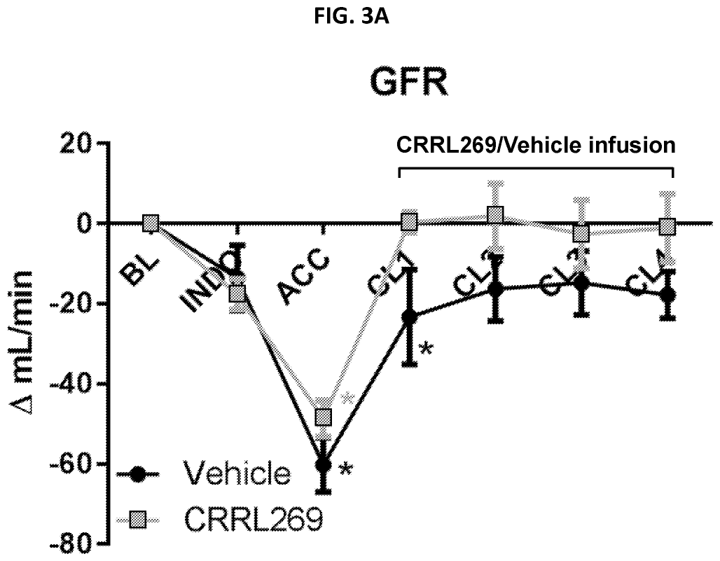

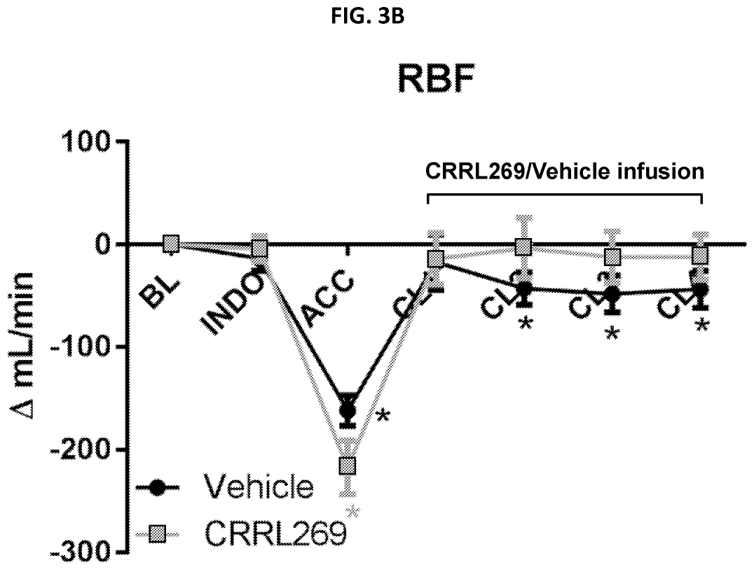

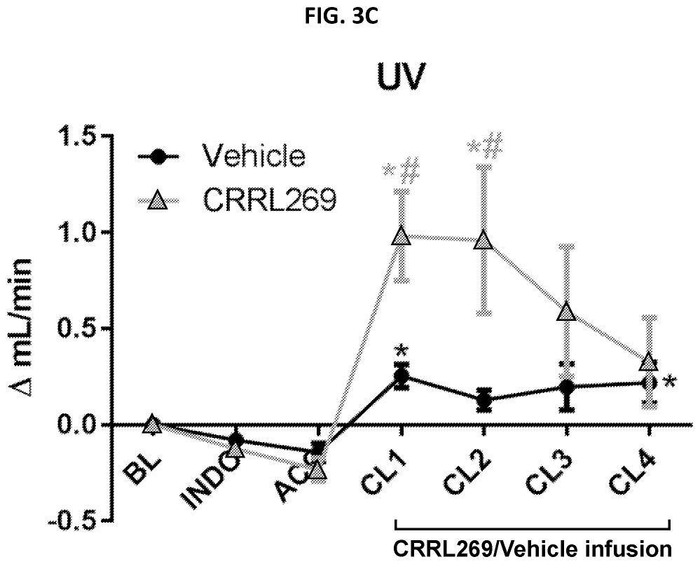

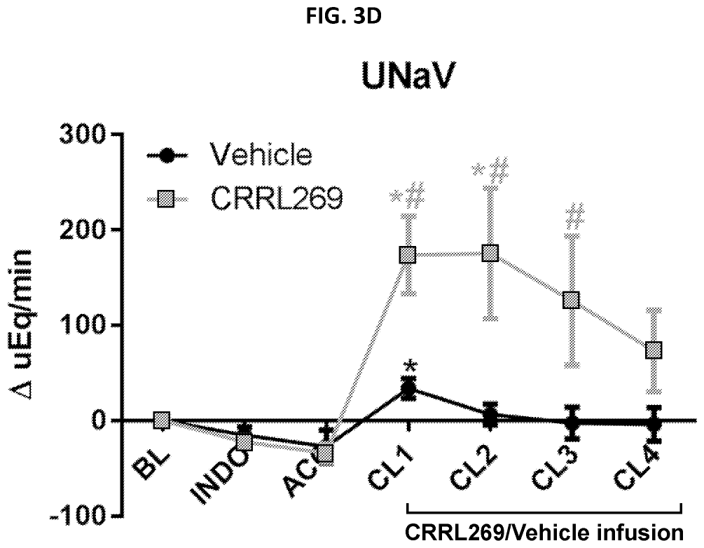

In vivo, continuous infusion for 2 hours with CRRL269 increased plasma, urinary and renal parenchyma cGMP levels, consistent with pGC-A activation (FIGS. 2A, 2B, 2C, and 2D). Results in vivo are presented as changes from baseline in the figures. Compared to vehicle, CRRL269 treatment markedly elevated cGMP values from CL1 to CL4. Notably, AKI induced by ACC greatly stimulated plasma ANGII levels compared to baseline, while CRRL269 inhibited plasma ANGII levels (FIG. 2D). Specifically, in the vehicle group, ANGII levels remained high during post-ischemia phases from CL1 to CL4, while CRRL269 infusion significantly reduced circulating ANGII levels. With ACC occluding of the suprarenal aorta, GFR was markedly reduced during ACC. Changes from baseline are shown in FIG. 3A. A marked reduction of RBF was also observed during ACC (FIG. 3B). Importantly, CRRL269 maintained GFR and RBF during postischemia phases, while vehicle did not preserve these two renal hemodynamic parameters (FIGS. 3A and 3B). Similarly, urine output (UV) and urinary sodium excretion (UNaV) were reduced by ACC while CRRL269 significantly induced diuresis and natriuresis compared to vehicle (FIGS. 3C and 3D), consistent with the renal protective actions observed with GFR and RBF.

### Example 3—Cardiovascular Function in Ischemic AKI Canines

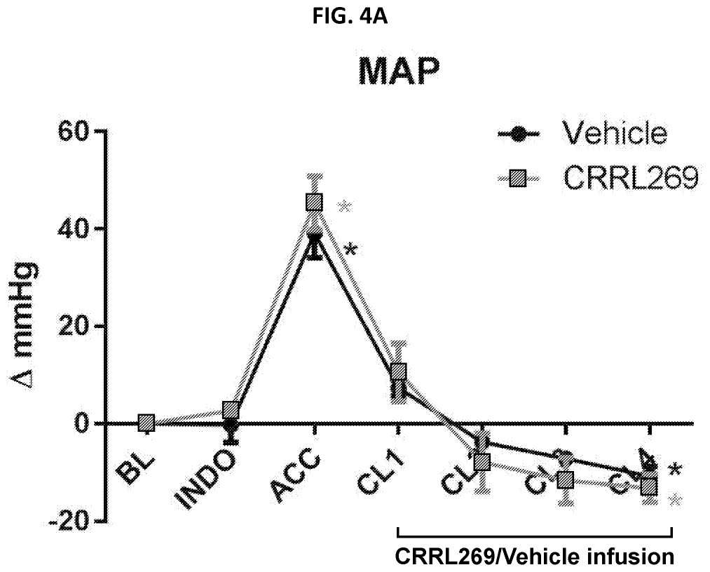

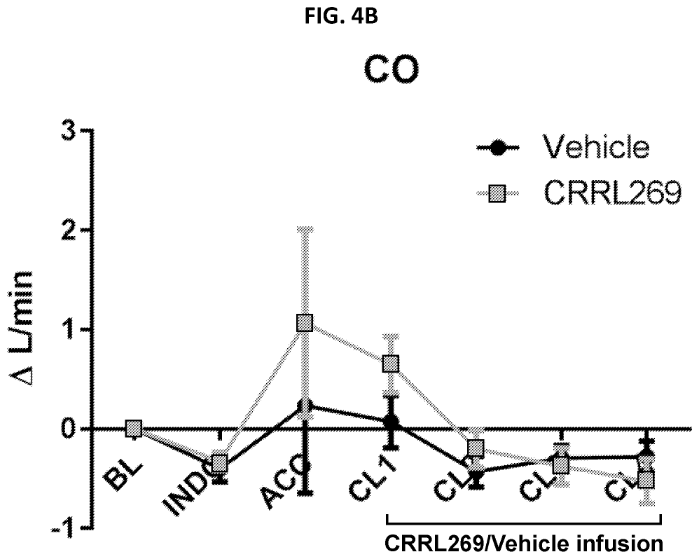

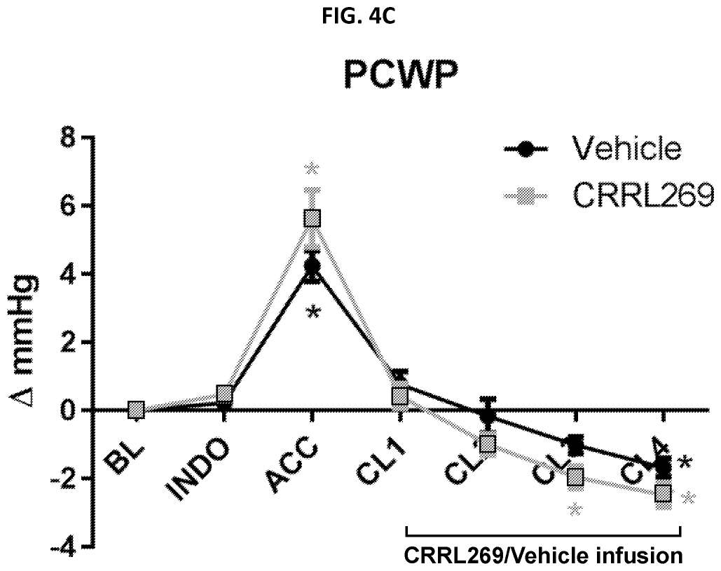

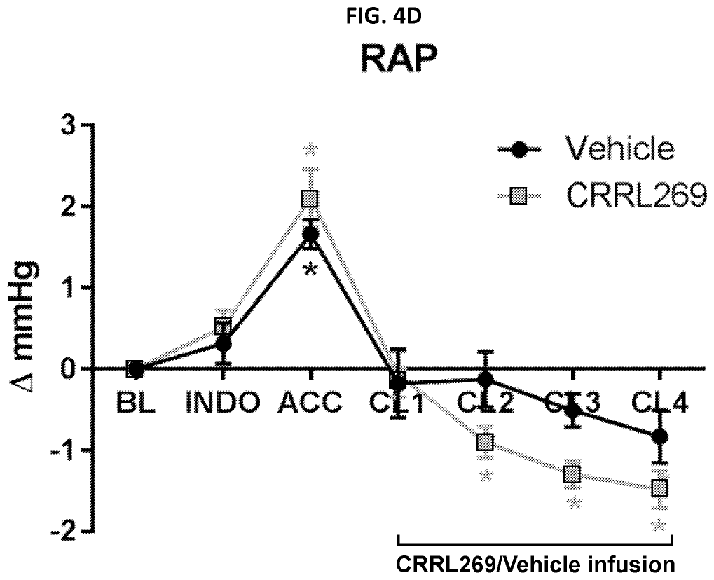

ACC resulted in a marked elevation of mean arterial pressure (MAP) that returned to baseline levels during post-ischemia reperfusion periods. Notably, CRRL269 induced similar BP effects compared with vehicle infusion (FIG. 4A), indicating that CRRL269 is not a hypotensive agent. A similar trend was observed in cardiac output (CO) (FIG. 4B). Right atrial pressure (RAP) and pulmonary capillary wedge pressure (PCWP) also were markedly elevated during renal ischemia. Of note, CRRL269 reduced RAP and PCWP after ischemia (FIGS. 4C and 4D). The results of cardiovascular function parameter assessment indicated that CRRL269 reduced cardiac filling pressure without a hypotensive response.

### Example 4—Renal Injury Ex Vivo Analysis

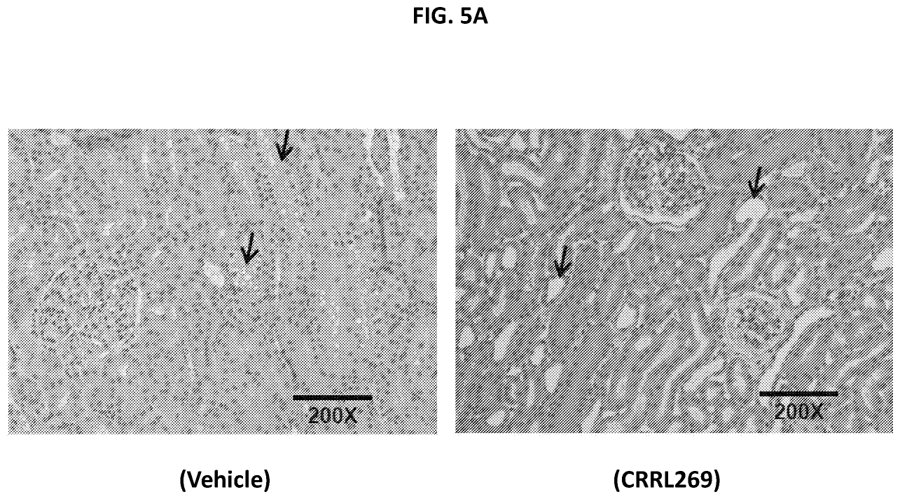

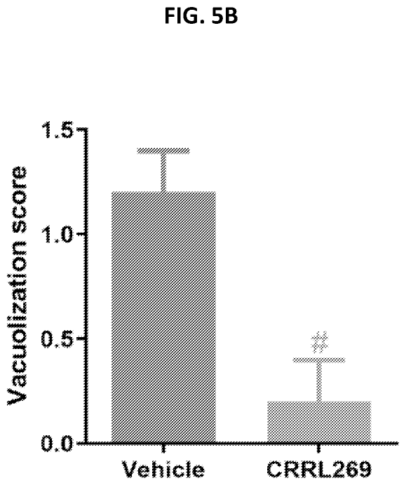

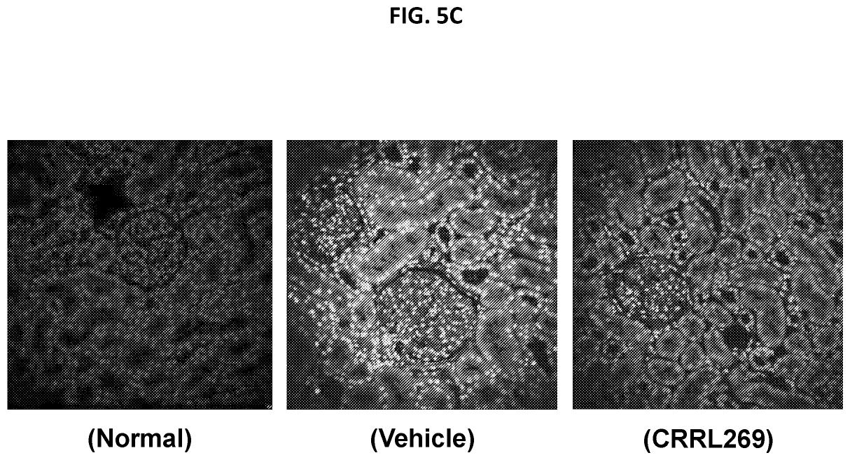

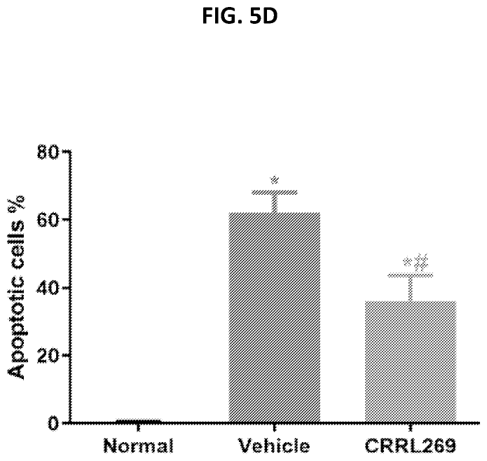

H&E staining indicated that the CRRL269 group presented with less vacuolization compared to vehicle consistent with less renal injury (FIGS. 5A and 5B). Additionally, ischemia/reperfusion markedly increased renal cortical apoptotic cell death compared to normal canine kidneys, as illustrated by TUNEL assay using con-focal microscopy (FIGS. 5C and 5D). Apoptotic cells were mostly tubular epithelial cells and glomerular mesangial cells. Importantly, CRRL269 treatment reduced apoptosis in the renal cortex as compared to vehicle.

### Example 5—H/R Mediated Apoptosis in HRPTC

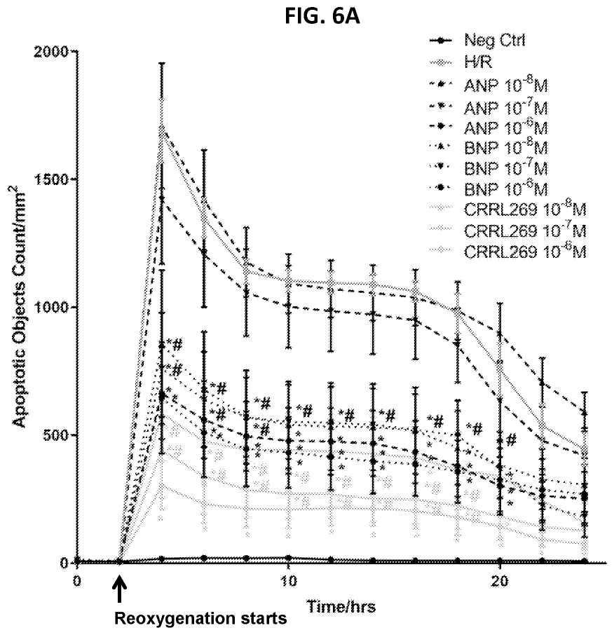

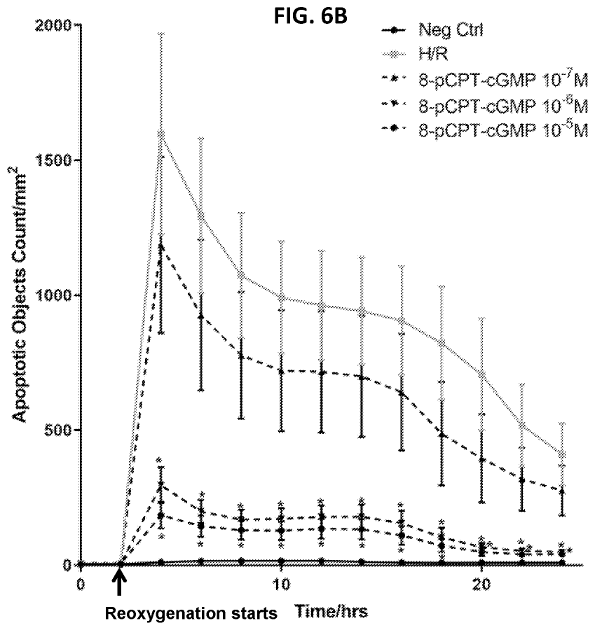

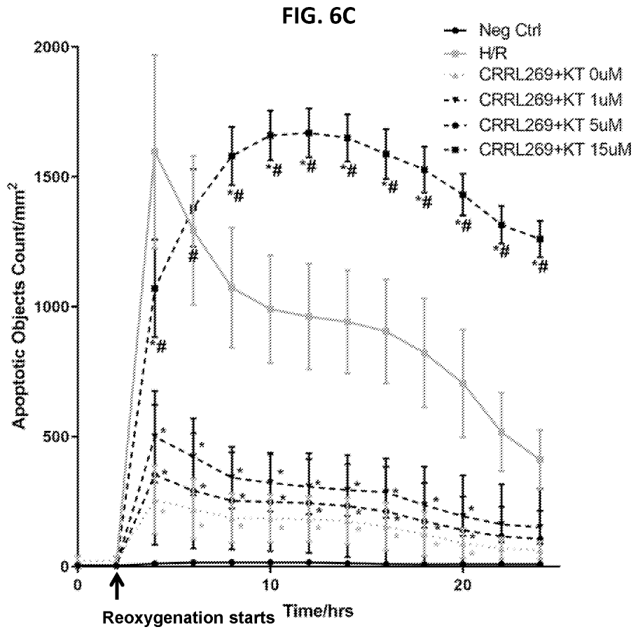

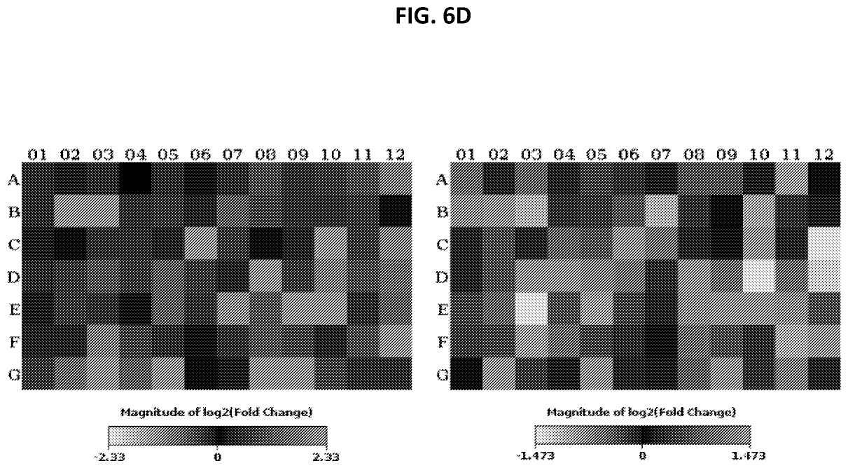

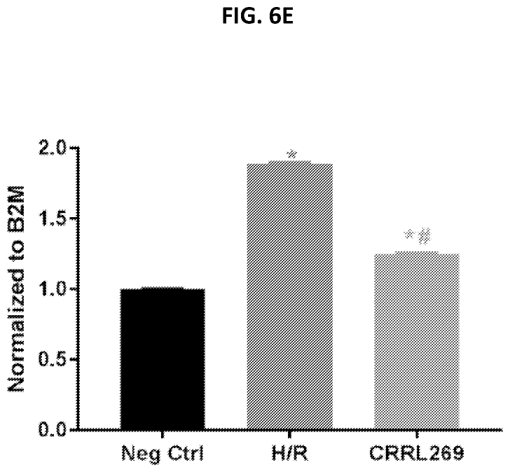

Recognizing the anti-apoptosis observed in renal tubular cells ex vivo by CRRL269, HRPTC were evaluated for in vitro apoptosis using a time-lapsed, live imaging system. H/R markedly stimulated apoptosis in HRPTC as evidenced by the sharp increase of apoptotic signals at 2 hours, in comparison with the negative control group (FIGS. 6A, 6B, and 6C). CRRL269 at concentrations of 10⁴ and 10¹ M significantly reduced apoptotic signals compared to the H/R group alone. More importantly, CRRL269 induced more robust anti-apototic effects compared to ANP, and a trend of superiority also was observed in comparison with BNP (FIG. 6A). The PKG specific analog 8-pCPT-cGMP at concentrations of 10⁻⁶ and 10⁻⁵ M mimicked the apoptosis inhibition observed with pGC-A activators (FIG. 6B). Further, PKG inhibitor KT-5823 abrogated the antiapoptotic effects induced by CRRL269 (FIG. 6C). In the human apoptosis pathway gene array, 33 out of 84 genes were up-regulated at least 2 fold by H/R stimulation. In contrast, 8 out of 84 genes (CASP7, FADD, CRADD, DAPK1, HGDC, BCL2L11, BIK, TNFRSF1A) were down-regulated at least 2 fold by CRRL269 treatment (FIG. 6D). Caspase7 gene (top gene, up and down-regulated in two groups) was chosen for verification by RT-PCR due to its critical function mediating apoptosis. The results showed that H/R increased expression of the Caspase7 gene by 1.89 fold, and CRRL269 treatment significantly reduced its expression level to 1.25 (FIG. 6E) (Holly et al., J Mol Cell Cardiol, 31:1709-1715, 1999).

### Example 6—Intracellular Ca2⁺ Levels Inhibited by pGC-A Activators Under ANGII Stimulation

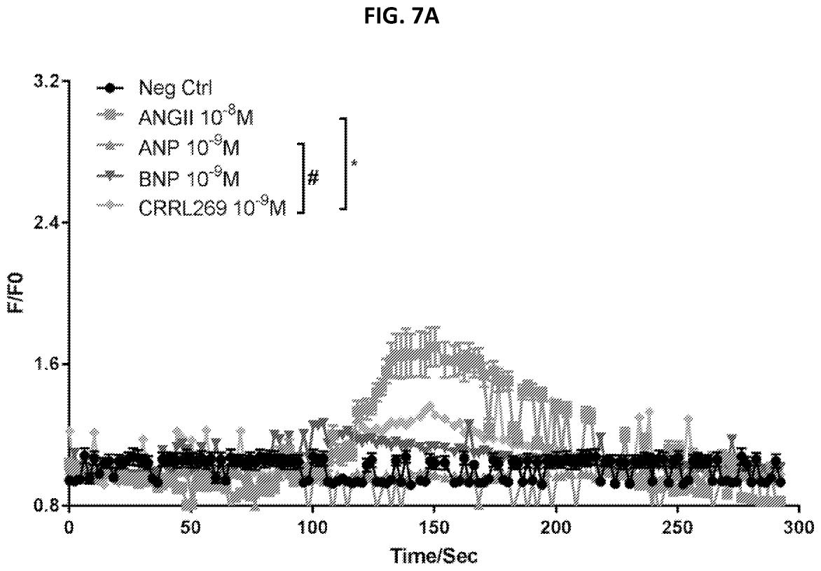

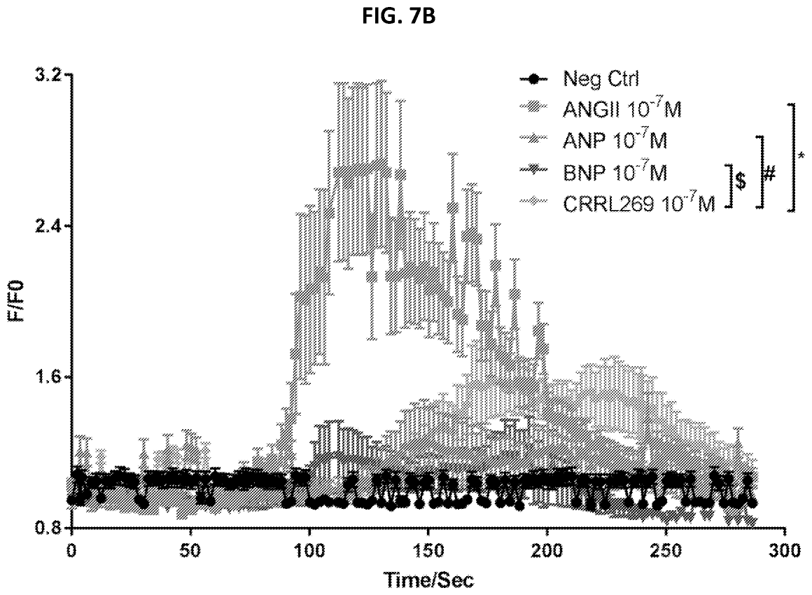

Intracellular Ca2⁺ levels in HASMC were increased by stimulation with the vasoconstrictor ANGII at 10⁻⁸ or 10⁻⁷ M (FIGS. 7A and 7B). Pretreatment with the pGC-A activator ANP, BNP, or CRRL269 at 10⁻⁹ or 10⁻⁷ M suppressed Ca2⁺ enhancement by ANGII. Notably, CRRL269 demonstrated less Ca2⁺ suppression than ANP and BNP.

### Example 7—CNP as a Compensatory Biomarker for AKI

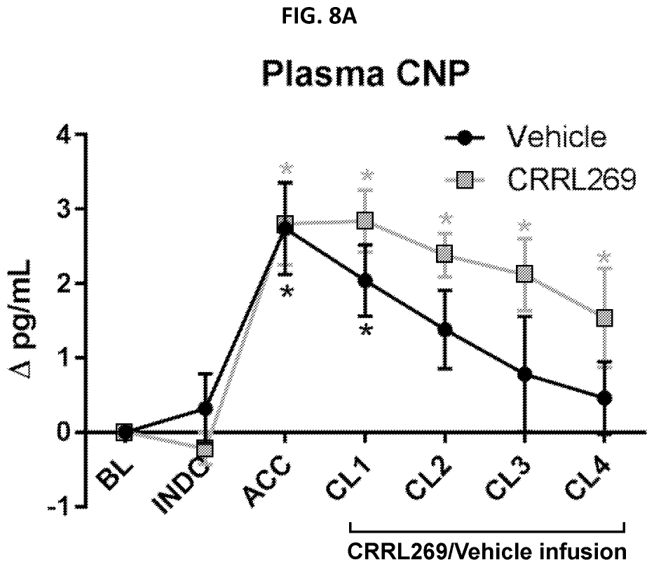

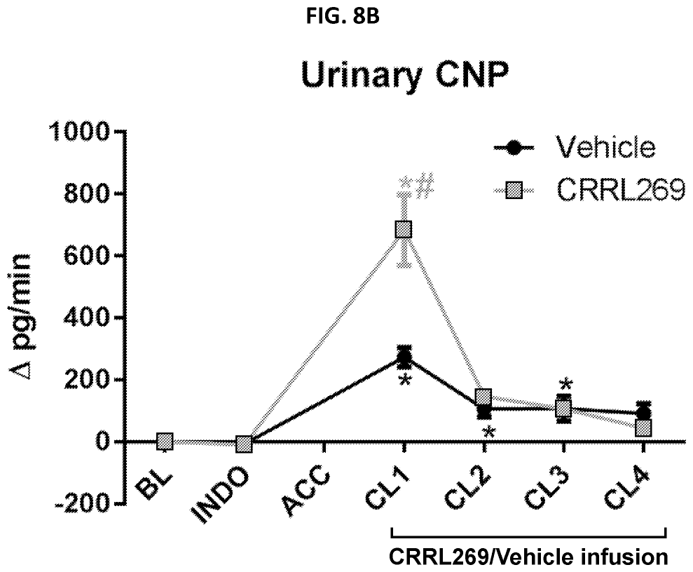

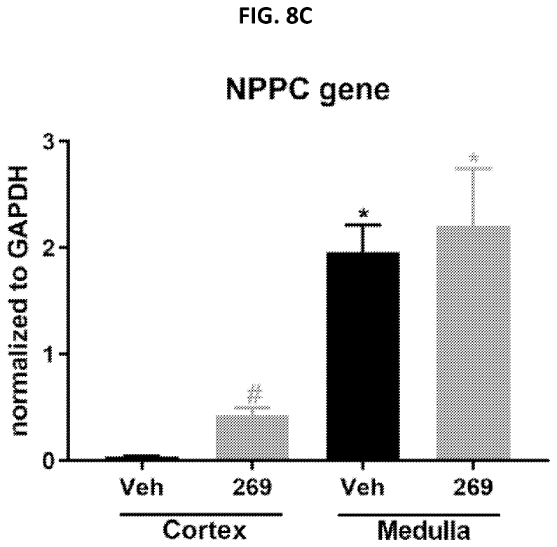

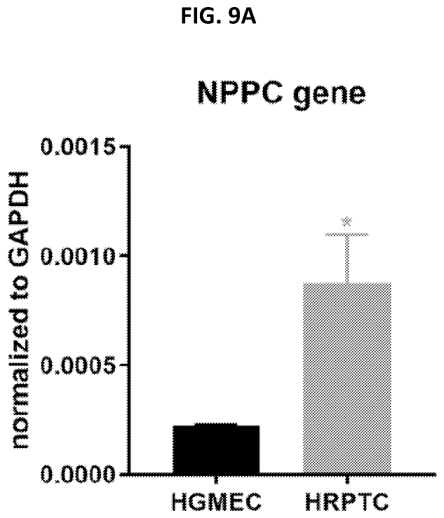

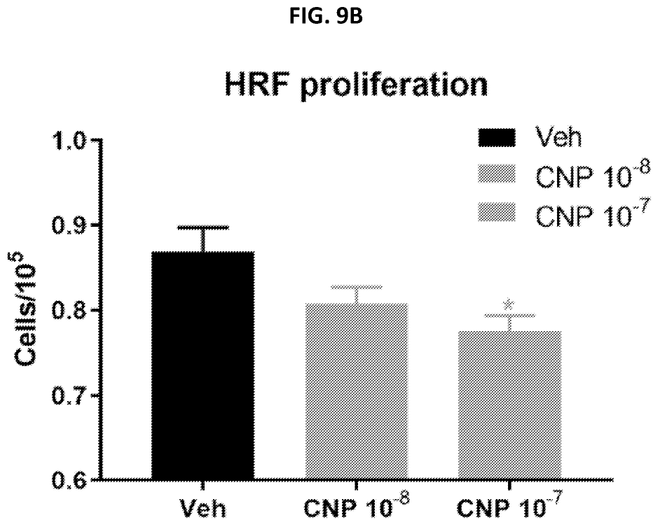

Plasma and urinary CNP levels were increased starting from the AKI reperfusion phase, with elevation in urinary CNP being more pronounced (FIGS. 8A and 8B). Infusion with CRRL269 increased urinary CNP levels compared to vehicle. Further, with RT-PCR, cortical NPPC gene expression levels, although lower compared to medullary expression, were elevated by CRRL269 treatment (FIG. 8C), reinforcing the CNP increasing effect of CRRL269. In vitro, HGMEC presented considerably lower NPPC mRNA levels than tubular HRPTC (FIG. 9A). Additionally, CNP treatment at 10⁻⁷ M suppressed human renal fibroblast (HRF) proliferation (FIG. 9B).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method comprising:
measuring the level of C-type natriuretic peptide (CNP) in a biological sample from a mammal;
identifying said mammal as having a CNP level that is elevated related to a corresponding control level of CNP; and
administering to said mammal a composition comprising a peptide that comprises, in order from N-terminus to C-terminus:
(a) the sequence set forth in SEQ ID NO:1 with three or less amino acid additions, deletions, substitutions, or combinations thereof,
(b) the sequence set forth in SEQ ID NO:2 with five or less amino acid additions, deletions, substitutions, or combinations thereof, and
(c) the sequence set forth in SEQ ID NO:3 with two or less amino acid additions, deletions, substitutions, or combinations thereof.

2. The method of claim 1, wherein said mammal is a human.

3. The method of claim 1, wherein said mammal is a human who is undergoing or has undergone cardiovascular surgery.

4. The method of claim 1, wherein said biological sample is a urine sample or a blood sample.

5. (canceled)

6. The method of claim 1, wherein said measuring comprises determining a level of CNP mRNA in said biological sample.

7. The method of claim 1, wherein said measuring comprises determining a level of CNP protein in said biological sample.

8. The method of claim 1, wherein said measuring comprises a radioimmunoassay.

9. The method of claim 1, comprising administering to said mammal a composition comprising a peptide having the sequence of SEQ ID NO:4.

10. The method of claim 1, comprising administering said peptide at a dose of about 0.001 to 0.01 μg/kg/minute for 24 to 96 hours.

11. The method of claim 1, comprising administering said peptide at a dose of about 0.005 μg/kg/minute for 48 to 72 hours.

12. The method of claim 1, further comprising administering to said mammal a diuretic.

13. A method for identifying a mammal in need of treatment for acute kidney injury (AKI), said method comprising:
measuring the level of CNP in a biological sample obtained from a mammal; and
identifying said mammal as having an elevated level of CNP in said biological sample, as compared to a control level of CNP,
thereby identifying said mammal as being in need of a therapy for AKI.

14. The method of claim 13, wherein said mammal is a human.

15. The method of claim 13, wherein said mammal is a human who is undergoing or has undergone cardiovascular surgery.

16. The method of claim 13, wherein said biological sample is a urine sample or a blood sample.

17. (canceled)

18. The method of claim 13, wherein said measuring comprises determining a level of CNP mRNA in said biological sample.

19. The method of claim 13, wherein said measuring comprises determining a level of CNP protein in said biological sample.

20. The method of claim 13, wherein said measuring comprises a radioimmunoassay.

21. The method of claim 13, wherein said therapy comprises a peptide comprising, in order from N-terminus to C-terminus:
(a) the sequence set forth in SEQ ID NO:1 with three or less amino acid additions, deletions, substitutions, or combinations thereof,
(b) the sequence set forth in SEQ ID NO:2 with five or less amino acid additions, deletions, substitutions, or combinations thereof, and
(c) the sequence set forth in SEQ ID NO:3 with two or less amino acid additions, deletions, substitutions, or combinations thereof.

22. The method of claim 13, wherein said therapy comprises a peptide having the amino acid sequence of SEQ ID NO:4.


SEQUENCE LISTING (8 sequences)

SEQ ID NO: 1 - protein, 10 aa, Homo sapiens
TAPRSLRRSS

SEQ ID NO: 2 - protein, 17 aa, Homo sapiens
CFGRKMDRISSSSGLGC

SEQ ID NO: 3 - protein, 5 aa, Homo sapiens
NSFRY

SEQ ID NO: 4 - protein, 32 aa, Artificial Sequence
  synthetic polypeptide
TAPRSLRRSSCFGRKMDRISSSSGLGCNSFRY

SEQ ID NO: 5 - protein, 126 aa, Homo sapiens
MHLSQLLACALLLTLLSLRPSEAKPGAPPKVPRTPPAEELAEPQAAGGGQKKGDKAPGGGGANLKGDRSRLLRDLRVDTKSRAAWARLLQEHPNARKYKGANKKGLSKGCFGLKLDRIGSMSGLGC

SEQ ID NO: 6 - protein, 22 aa, Homo sapiens
GLSKGCFGLKLDRIGSMSGLGC

SEQ ID NO: 7 - DNA, 382 bp, Homo sapiens
atgcatctctcccagctgctggcctgcgccctgctgctcacgctgctctccctccggccctccgaagccaagcccggggcgccgccgaaggtcccgcgaaccccgccggcagaggagctggccgagccgcaggctgcgggcggcggtcagaagaagggcgacaaggctcccgggggcgggggcgccaatctcaagggcgaccggtcgcgactgctccgggacctgcgcgtggacaccaagtcgcgggcagcgtgggctcgccttctgcaagagcaccccaacgcgcgcaaatacaaaggagccaacaagaagggcttgtccaagggctgcttcggcctcaagctggaccgaatcggctccatgagcggcctgggatgttagt

SEQ ID NO: 8 - DNA, 66 bp, Homo sapiens
ggcttgtccaagggctgcttcggcctcaagctggaccgaatcggctccatgagcggcctgggatgt